(19) United States
(12) Patent Application Publication
(10) Pub. No.: US 2023/0263694 A1
(43) Pub. Date: Aug. 24, 2023

(54) COSMETIC DEVICE

(71) Applicant: LG HOUSEHOLD & HEALTH CARE LTD., Seoul (KR)

(72) Inventors: Chang Hwan HYUN, Seoul (KR); Kyung Won KIM, Seoul (KR)

(73) Assignee: LG HOUSEHOLD & HEALTH CARE LTD., Seoul (KR)

(21) Appl. No.: 17/922,655

(22) PCT Filed: Jan. 12, 2021
(86) PCT No.: PCT/KR2021/000396

§ 371 Date: Nov. 1, 2022

(30) Foreign Application Priority Data
Jun. 19, 2020 (KR) 10-2020-0075178

(51) Int. Cl. A61H 7/00

(52) U.S. Cl. CPC A61H 7/005; A61H 7/007; A61H 2007/009; A61H 2201/1215; A61H 2201/1695

(57) ABSTRACT

The present disclosure relates to a cosmetic device and a cosmetic device which massages skin while a plurality of massage tools rotate eccentrically by operation of a motor is characterized by including an abnormality detector configured to generate an abnormal signal when an abnormality occurs in the motor or each of the plurality of massage tools, and a reverse unit configured to operate according to the abnormal signal, wherein the reverse unit is configured to perform an auto-reverse function of eccentrically rotating the eccentric rotation of the plurality of massage tools at a predetermined angle in an opposite direction.

### TECHNICAL FIELD

The present disclosure relates to a cosmetic device, and more particularly, to a cosmetic device with a massage tool alignment function that diversifies the massage area pattern when a plurality of massage tools rotate eccentrically to massage the skin, and an auto reverse function that releases restraints when restraints caused by external forces occur on the plurality of massage tools.

### BACKGROUND ART

In general, a cosmetic device for massage for relaxing the skin or releasing tight muscles are provided in various types of products.

Recently, a cosmetic device that massages the face, arms, legs, and other parts of the human body while a plurality of massage tools similar to the shape of a finger rotate eccentrically has been in the spotlight.

Such a cosmetic device is operated as if kneading by hand while changing a plurality of massage tools to a minimum and maximum area.

However, in the existing eccentric rotating type cosmetic device, while the kneading function may be implemented as a plurality of massage tools are eccentric at a certain angle and rotate eccentrically at a constant speed, there is a limitation that the twisting function may not be implemented and there is a limitation in that the pattern constituting the massage area is monotonous.

In addition, the existing cosmetic device does not have a function to release the restraint when a restraint occurs due to an external force such as a finger pinched in a plurality of massage tools, causing damage to the device due to an overload applied to the driving unit, and if a finger is pinched, the user cannot easily remove the finger causing a problem in that the user has to suffer until the area becomes wide.

### DISCLOSURE OF THE INVENTION

### Technical Goals

The present disclosure has been proposed to solve the problems of the related art as described above, and an object of the present disclosure is to provide a cosmetic device with a massage tool alignment function that diversifies the massage area pattern when a plurality of massage tools rotate eccentrically to massage the skin.

In addition, an object of the present disclosure is to provide a cosmetic device with an auto reverse function that releases restraints when restraints caused by external forces occur on the plurality of massage tools.

### Technical Solutions

A cosmetic device according to a technical idea of the present disclosure is a cosmetic device which massages skin while a plurality of massage tools rotate eccentrically by operation of a motor, characterized by including an abnormality detector configured to generate an abnormal signal when an abnormality occurs in the motor or each of the plurality of massage tools, and a reverse unit configured to operate according to the abnormal signal, wherein the reverse unit is configured to perform an auto-reverse function of eccentrically rotating the eccentric rotation of the plurality of massage tools at a predetermined angle in an opposite direction.

Specifically, the reverse unit may be configured to receive the abnormal signal to check a restraint state of the plurality of massage tools for a first time, stop the eccentric rotation of the plurality of massage tools for a second time, cause the eccentric rotation of the plurality of massage tools to be eccentrically rotated in a reverse direction for a third time, stop the eccentric rotation of the plurality of massage tools again for a fourth time, and cause the eccentric rotation of the plurality of massage tools to be eccentrically rotated in a reverse direction for a fifth time and then the plurality of massage tools to be re-operated in forward rotation.

Specifically, the plurality of massage tools may be respectively connected to a transmission gear which is built in a gearbox connected to the motor and individually controlled, and may be individually operated according to control of an eccentric rotation speed and an eccentric rotation direction of the transmission gear.

Specifically, when massaging skin while rotating eccentrically, the plurality of massage tools may compositely implement a massage function of kneading, a massage function of rubbing, and a massage function of twisting in various non-periodic massage area patterns.

Specifically, the cosmetic device may further include a plurality of shaft gears to which the plurality of massage tools are coupled, respectively, and a plurality of clutches provided separately between the gearbox and the plurality of shaft gears.

Specifically, the plurality of massage tools may be individually regulated by the plurality of clutches, and the regulated corresponding massage tools may not rotate eccentrically.

Specifically, the plurality of shaft gears may be formed in a same gear ratio, may be formed in different gear ratios, or may be formed in at least two or more stages with gears having different gear ratios.

Specifically, each of the plurality of shaft gears may be connected to each of the plurality of clutches, respectively, when formed in the same gear ratio, the plurality of massage tools may have the same eccentric rotation speed, and when formed in the different gear ratios or formed in the at least two or more stages with gears having different gear ratios, the plurality of massage tools may have different eccentric rotation speeds.

Specifically, the cosmetic device may further include a pulse width modulator configured to apply an electronic muscle stimulation (EMS) to the plurality of massage tools, and the pulse width modulator may be configured to control EMS intensity of the plurality of massage tools while varying a voltage and a frequency.

Specifically, the pulse width modulator may be configured to perform a first EMS for a first time while varying a frequency in a state where a first voltage is applied, perform a second EMS for a second time while varying the frequency in a state where a second voltage higher than the first voltage is applied, perform a third EMS for a third time while varying the frequency in a state where a third voltage higher than the second voltage is applied, and perform the first EMS again after stopping the EMS for a predetermined period of time.

Specifically, the plurality of massage tools may be provided inclined by a predetermined angle, and may rotate eccentrically after their initial positions are aligned with a center of the device.

### Advantageous Effects

A cosmetic device according to the present disclosure is configured to have an auto reverse function that rotates eccentrically at a certain angle in the opposite direction to release the restraint when the restraint by an external force occurs in a plurality of massage tools, so that the damage to the device due to excessive overload or safety in use may be secured.

In addition, the cosmetic device according to the present disclosure is configured to be able to individually control the eccentric rotation speed, eccentric rotation direction, and the presence or absence of eccentric rotation of each of the plurality of massage tools, so that when the plurality of massage tools rotate eccentrically to massage the skin, by adding the massage area pattern to the basic area pattern, various non-periodic massage area patterns can be realized, thereby maximizing massage efficiency.

### BRIEF DESCRIPTION OF DRAWINGS

FIG. 1 is an exploded perspective view of a cosmetic device according to an embodiment of the present disclosure.

FIG. 2 is a front view of a cosmetic device according to an embodiment of the present disclosure.

FIG. 3 is a plan perspective view of a cosmetic device according to an embodiment of the present disclosure.

FIG. 4 is a rear perspective view of a cosmetic device according to an embodiment of the present disclosure.

FIG. 5 is a block diagram to explain a cosmetic device according to an embodiment of the present disclosure.

FIG. 6 is a block diagram to explain a driving unit of FIG. 5.

FIG. 7 is a flowchart to explain an operation of a cosmetic device according to an embodiment of the present disclosure.

FIGS. 8 to 14 are diagrams to explain a physical massage function of a cosmetic device according to an embodiment of the present disclosure.

FIG. 15 is a waveform diagram to explain an electric massage function of a cosmetic device according to an embodiment of the present disclosure.

FIGS. 16 to 18 are diagrams to explain various external shapes of a cosmetic device according to an embodiment of the present disclosure.

### BEST MODE FOR CARRYING OUT THE INVENTION

The objects, specific advantages and novel features of the present disclosure will become more apparent from the following detailed description and preferred embodiments taken in conjunction with the accompanying drawings. In the present specification, in adding reference numerals to the components of each drawing, it should be noted that only the same components are given the same numeral as possible even though they are indicated on different drawings. In addition, in describing the present disclosure, if it is determined that a detailed description of a related known art may unnecessarily obscure the subject matter of the present invention, the detailed description thereof will be omitted.

Hereinafter, preferred embodiments of the present disclosure will be described in detail with reference to the accompanying drawings.

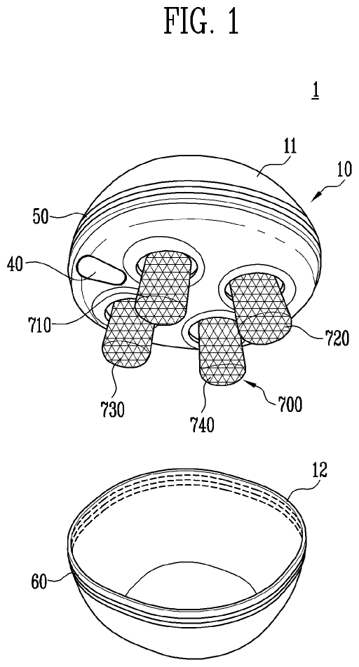

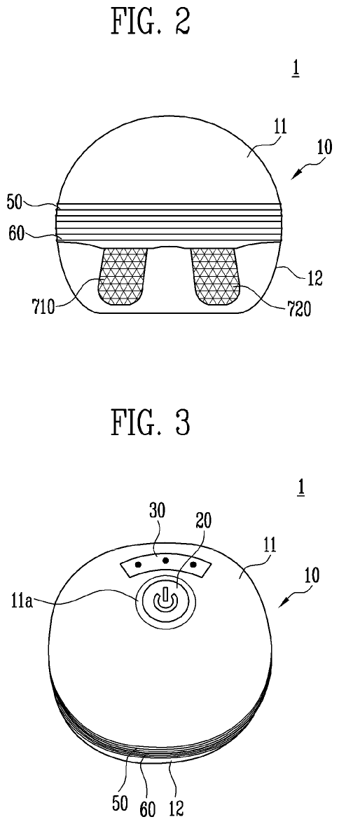

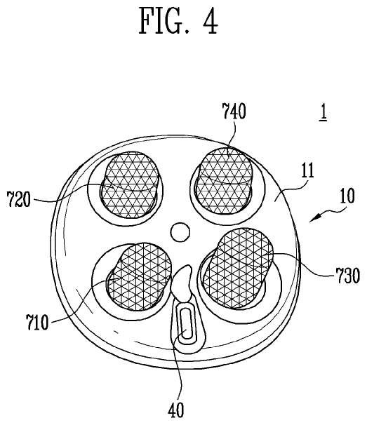

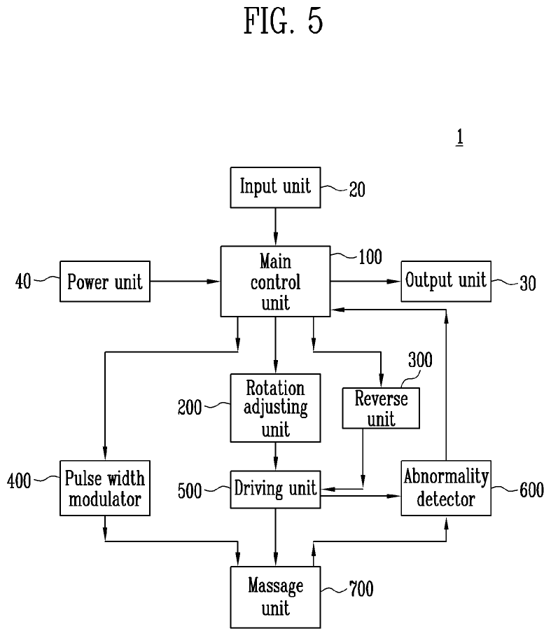

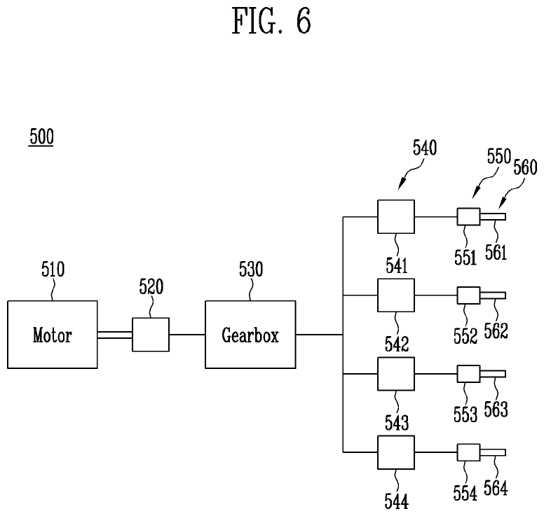

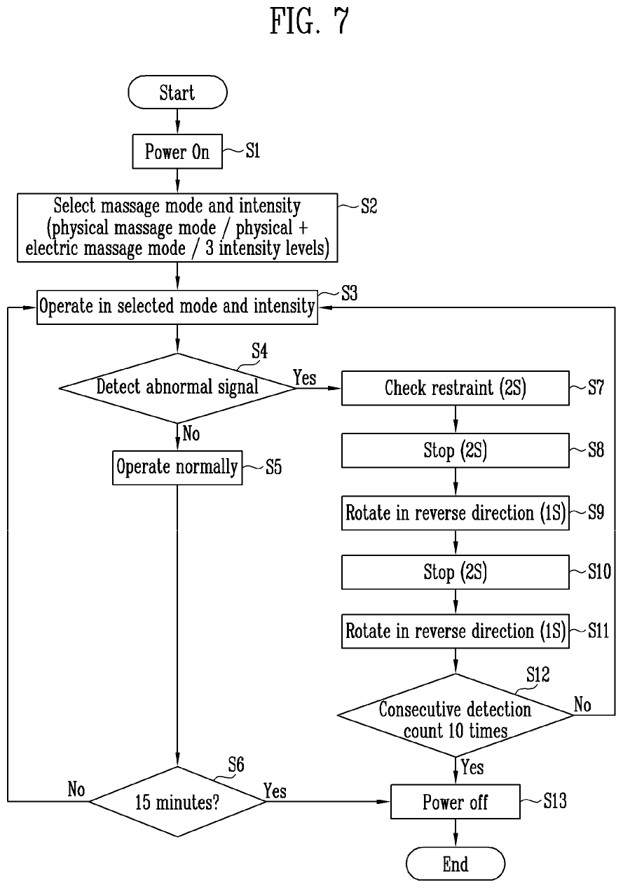

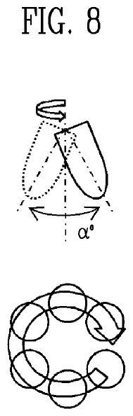

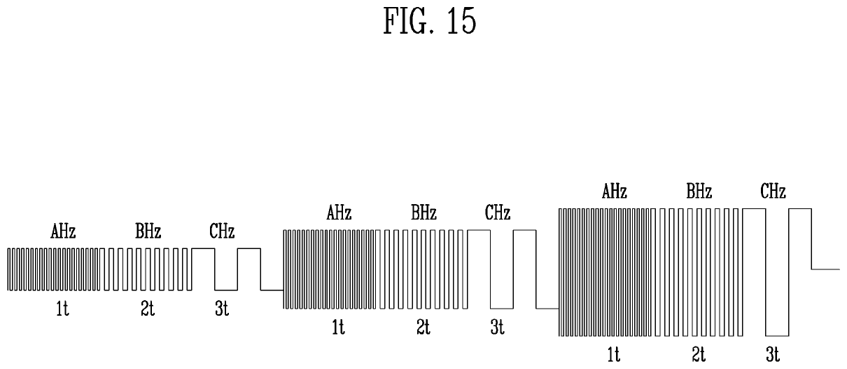

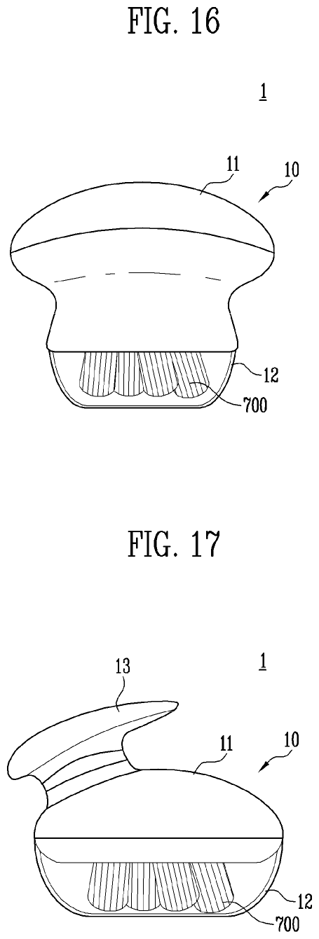

FIG. 1 is an exploded perspective view of a cosmetic device according to an embodiment of the present disclosure, FIG. 2 is a front view of a cosmetic device according to an embodiment of the present disclosure, FIG. 3 is a plan perspective view of a cosmetic device according to an embodiment of the present disclosure, FIG. 4 is a rear perspective view of a cosmetic device according to an embodiment of the present disclosure, FIG. 5 is a block diagram to explain a cosmetic device according to an embodiment of the present disclosure, FIG. 6 is a block diagram to explain a driving unit of FIG. 5, FIG. 7 is a flowchart to explain an operation of a cosmetic device according to an embodiment of the present disclosure, FIGS. 8 to 14 are diagrams to explain a physical massage function of a cosmetic device according to an embodiment of the present disclosure, FIG. 15 is a waveform diagram to explain an electric massage function of a cosmetic device according to an embodiment of the present disclosure, and FIGS. 16 to 18 are diagrams to explain various external shapes of a cosmetic device according to an embodiment of the present disclosure.

Referring to FIGS. 1 to 6, a cosmetic device 1 according to an embodiment of the present disclosure is an eccentric rotation type massage device that allows a plurality of massage tools to massage the skin while rotating and eccentrically rotating at the same time, and may be configured to include a body 10, an input unit 20, an output unit 30, a power unit 40, a main control unit 100, a rotation adjusting unit 200, a reverse unit 300, a pulse width modulator 400, a driving unit 500, an abnormality detector 600, and a massage unit 700.

The body 10 may include a housing 11 and a cover 12.

The housing 11 may provide a space for accommodating various parts, and the overall shape may be a dome shape. The upper portion has a hemispherical shape so that a user can conveniently hold it by hand, and an input unit 20 and an output unit 30 may be provided. The lower portion forms a flat plate shape, and a massage unit 700 may be provided.

The cover 12 is detachable from the lower portion of the housing 11, and can protect the massage unit 700. The cosmetic device 1 is stored in a state in which the cover 12 is in close contact with the floor, and for this purpose, the lower end of the cover 12 forms a flat surface.

The housing 11 is a part held by the user's hand when massaging, giving an aesthetic feeling, and may be formed in various shapes for the convenience of the user.

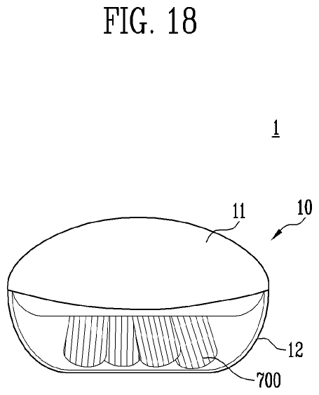

The housing 11 may be formed in a hemispherical shape as shown in FIG. 2, may be formed in a mushroom shape as shown in FIG. 16, may be formed such that a separate handle 13 is provided in a flat hemispherical shape as shown in FIG. 17, or may be formed in a turtle back shape as shown in FIG. 18.

As such, the upper portion of the housing 11 has a curved surface so that a user can conveniently hold it by hand, and may be formed in various shapes.

If the surface of the housing 11 is formed smoothly, the cosmetic device 1 may be separated from the hand during massage, and there may be difficulties when opening and closing the cover 12, and in order to prevent this, a first grip unit 50 may be provided in the housing 11.

The first grip unit 50 may be formed to have a predetermined width at a portion contacting the tip of a finger when the user holds it by hand, for example, at a portion contacting the cover 12 with the largest radius of the housing 11. The first grip unit 50 may be formed of a spiral groove and a protrusion.

A second grip unit 60 identical to or similar to the first grip unit 50 may be provided on the cover 12 as well. The second grip unit 60 may conveniently open and close the cover 12 together with the first grip unit 50.

The input unit 20 is provided on the upper portion of the housing 11 and is connected to the main control unit 100, and may select and input power and various modes. The input unit 20 may be provided in the form of a button, and functions such as power on/off, physical massage mode, electric massage mode, massage intensity adjustment mode, rotation speed and eccentric rotation speed adjustment mode of the massage tool can be selected.

In addition, the periphery of the input unit 20 may be formed in a shape to protect the input unit 20. As shown in FIG. 3, a protective rim 11a may be provided along the periphery of the input unit 20. The protective rim 11a may be formed by protruding the housing 11 around the input unit 20 so that the input unit 20 does not protrude from the surface of the housing 11 to the outside.

The output unit 30 is provided on the upper portion of the housing 11 and is connected to the main control unit 100, and can visualize and output various types of information. The output unit 30 may be provided in the form of an LED lamp, a display window, etc., and may visually provide information about how and in which mode the cosmetic device 1 is currently operating to the user.

The power unit 40 is provided on the lower portion of the housing 11 and is connected to the main control unit 100, and may supply power. The power unit 40 may be a USB type C that can be connected to an external power source. In addition, the power unit 40 may be a battery (not shown) built into the housing 11.

The main control unit 100 may control the rotation adjusting unit 200, the reverse unit 300, the pulse width modulator 400, and the driving unit 500 according to input information of the input unit 20.

Here, the rotation adjusting unit 200 controls the driving unit 500 to individually adjust the eccentric rotation speed and rotation speed of the first, second, third, and fourth massage tools 710, 720, 730, 740 of the massage unit 700 to be described later, and enables the physical massage function to be performed. The rotation adjusting unit 200 can individually control the eccentric rotation speed and rotation speed of the first, second, third, and fourth massage tools 710, 720, 730, and 740 in three steps while varying the RPM, but it not limited thereto.

The pulse width modulator 400 allows a micro current such as an electronic muscle stimulation (EMS) to flow through the first, second, third, and fourth massage tools 710, 720, 730, and 740, and enables an electric massage function to be performed. The pulse width modulator 400 may control the electrical stimulation intensity of the first, second, third, and fourth massage tools 710, 720, 730, and 740 in three steps while varying the voltage and frequency, and is not limited thereto.

In addition, the main control unit 100 may control the reverse unit 300 according to the abnormal signal information of the abnormality detector 600. The abnormal signal of the abnormality detector 600 is generated when the overcurrent generated by the overload applied to the motor 510 becomes greater than or equal to a preset value, and the preset value of the overcurrent varies depending on the RPM control of the rotation adjusting unit 200.

Here, when restraint by external force occurs such as a finger being pinched in at least one of the first, second, third, and fourth massage tools 710, 720, 730, and 740 of the massage unit 700 while normally eccentrically rotating, the reverse unit 300 enables the auto-reverse function to rotate eccentrically at a predetermined angle in the opposite direction to release the restraint.

The driving unit 500 enables the first, second, third, and fourth massage tools 710, 720, 730, and 740 to rotate forward or reverse by the rotation adjusting unit 200 or the reverse unit 300, and in addition, may include a motor 510, a driving gear 520, a gearbox 530, a clutch unit 540, a shaft gear unit 550, and a drive shaft unit 560 in order to be able to individually adjust the eccentric rotation speed and eccentric rotation direction of the first, second, third, and fourth massage tools 710, 720, 730 and 740 by the rotation adjusting unit 200.

The driving gear 520 may transmit the power of the motor 510 to the driving gear 520.

The gearbox 530 is connected to the driving gear 520, and various gears (for example, sun gear, ring gear, planetary gear, worm gear, transmission gear, etc.) for transmitting power required for eccentric rotation of the first, second, third, and fourth drive shafts 561, 562, 563 and 564 of the drive shaft unit 560 are built therein. Since the gearbox 530 may be a known box, a detailed description thereof will be omitted herein. However, the gearbox 530 has a built-in gear configuration that allows the eccentric rotation speed and eccentric rotation direction of the first, second, third, and fourth massage tools 710, 720, 730 and 740 to be individually adjusted.

The clutch unit 540 may include first, second, third, and fourth clutches 541, 542, 543 and 544 that regulate the power transmitted from the gearbox 530 to the first, second, third, and fourth drive shafts 561, 562, 563 and 564. These first, second, third and fourth clutches 541, 542, 543 and 544 can be operated individually, and accordingly, may individually regulate the eccentric rotation of the first, second, third, and fourth drive shafts 561, 562, 563, and 564 to which the first, second, third, and fourth massage tools 710, 720, 730, and 740 are coupled.

The shaft gear unit 550 is connected to each of the first, second, third and fourth clutches 541, 542, 543 and 544, and may include first, second, third, and fourth shaft gears 551, 552, 553 and 554 provided with first, second, third, and fourth drive shafts 561, 562, 563 and 564, respectively. The first, second, third, and fourth shaft gears 551, 552, 553 and 554 may be configured to adjust the eccentric rotation speed of the first, second, third, and fourth drive shafts 561, 562, 563 and 564.

In other words, when the first, second, third, and fourth shaft gears 551, 552, 553 and 554 are formed in the same gear ratio, the eccentric rotation speed of the first, second, third, and fourth drive shafts 561, 562, 563 and 564 will be the same, but it is possible to make the eccentric rotation speed of the first, second, third, and fourth drive shafts 561, 562, 563 and 564 be different by forming in different gear ratios, or by allowing different stages to be selected by the first, second, third, and fourth clutches 541, 542, 543 and 544 by forming in at least two or more stages with gears having different gear ratios.

The drive shaft unit 560 may include first, second, third and fourth drive shafts 561, 562, 563 and 564 so that each of the first, second, third and fourth massage tools 710, 720, 730 and 740 can be coupled thereto.

In the present embodiment described above, although it has been described as a configuration in which each of the motor 510 and the gearbox 530 of the driving unit 500 is one, and a plurality of first, second, third, and fourth massage tools 710, 720, 730 and 740 are individually connected to one gearbox 530, the gearbox 530 may be provided in plurality to be connected to each or two or three of the first, second, third, and fourth massage tools 710, 720, 730 and 740, and the motor 510 may also be provided in plurality. As such, when the motor 510 and the gearbox 530 are provided in plurality instead of one, the capacity of the motor 510 and the configuration of the gears built into the gearbox 530 are different from those in the case where one is provided.

When an abnormality occurs in each of the first, second, third, and fourth massage tools 710, 720, 730 and 740 of the motor 510 of the driving unit 500 or the massage unit 700, the abnormality detector 600 may transmit an abnormality signal to the main control unit 100. Here, the main control unit 100 may cause the reverse unit 300 to operate according to the transmitted abnormal signal.

Specifically, when restraint by external force occurs such as a finger being pinched in at least one of the first, second, third, and fourth massage tools 710, 720, 730, and 740 while normally eccentrically rotating, the eccentric rotation is limited and the motor 510 is overloaded, and when the overcurrent generated at this time exceeds the preset value, the abnormality detector 600 may detect this and transmits an abnormal signal to the main control unit 100, or may detect that the first, second, third, and fourth massage tools 710, 720, 730 and 740 are stopped and transmit an abnormal signal to the main control unit 100.

The massage unit 700 may be provided on the lower portion of the housing 11 and coupled to the shaft gear unit 550 of the driving unit 500. Four massage units 700 may be provided as a plurality of massage tools, for example, first, second, third, and fourth massage tools 710, 720, 730 and 740. Although it is described that the number of massage tools is four in this embodiment, it may be provided in various numbers such as two, three, or five.

Each of the first, second, third, and fourth massage tools 710, 720, 730 and 740 may be coupled to each of the first, second, third, and fourth shaft gears 551, 552, 553 and 554 of the shaft gear unit, and may be eccentrically coupled at an angle of α° with respect to each of the shaft gears 551, 552, 553 and 554. As a result, the first, second, third, and fourth massage tools 710, 720, 730 and 740 eccentrically rotate at the same time as they rotate to massage the skin in various massage area patterns. In other words, the first, second, third, and fourth massage tools 710, 720, 730 and 740 may perform a physical massage function. Preferably, it is inclinedly coupled at an angle of 12° so that the first, second, third, and fourth massage tools 710, 720, 730 and 740 rotate eccentrically, and implement continuous motion of twisting the skin from side to side, widening, and narrowing, thereby inducing a massage effect simulating finger movement.

In addition, each of the first, second, third, and fourth massage tools 710, 720, 730 and 740 of this embodiment may have a protrusion or a polygonal angled form rather than flattening so as to increase the massage effect even when rotating.

The physical massage functions of the first, second, third, and fourth massage tools 710, 720, 730 and 740 will be described with reference to FIGS. 8 to 14. Hereinafter, the operating radius may be a physical massage area (skin massage trajectory) as shown below in each figure.

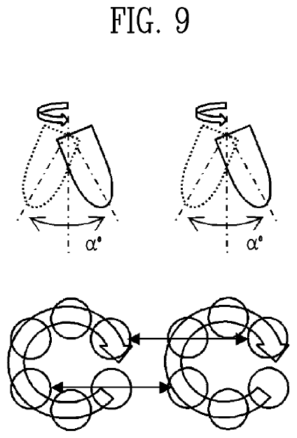

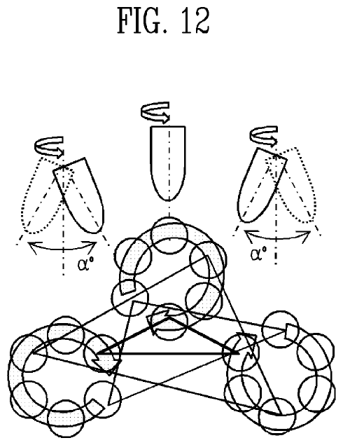

In this embodiment, the physical massage function in the case where the cosmetic device 1 is composed of four massage tools 710, 720, 730 and 740 will be described with reference to FIGS. 8 to 14, in the case of FIGS. 9 to 11, the same or similar physical massage function may be implemented even for a cosmetic device composed of two massage tools, and in the case of FIG. 12, the same or similar physical massage function may be implemented in a cosmetic device composed of three massage tools.

FIG. 8 shows a basic operating radius structure when the first, second, third, and fourth massage tools 710, 720, 730 and 740 each rotate eccentrically in the forward direction in a state eccentric at an angle of α°. This basic operating radius is the same for eccentric rotation in the reverse direction. Each of the first, second, third, and fourth massage tools 710, 720, 730 and 740 may rotate with eccentric rotation, and the rotation direction may be in the forward or reverse direction.

FIG. 9 shows the operating radius structure of the two adjacent massage tools in the first, second, third, and fourth massage tools 710, 720, 730 and 740 when having the same initial positions and eccentrically rotating in the same direction. Here, it can be seen that horizontal movements in the same width occur in two massage tools.

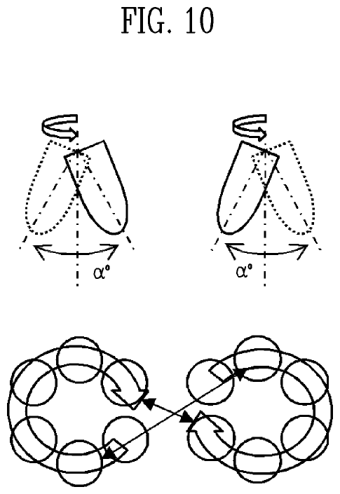

FIG. 10 shows the operating radius structure of the two adjacent massage tools in the first, second, third, and fourth massage tools 710, 720, 730, and 740 when having the symmetric initial positions and eccentrically rotating in the same direction. Here, it can be seen that vertical and horizontal movements in different widths and torsion occur in two massage tools.

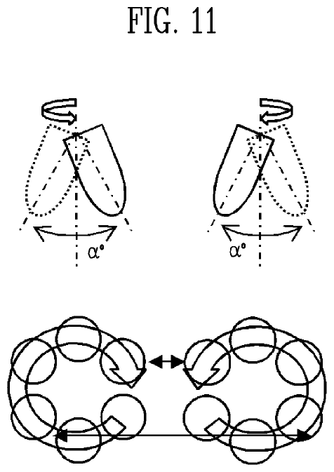

FIG. 11 shows the operating radius structure of the two adjacent massage tools in the first, second, third, and fourth massage tools 710, 720, 730 and 740 when having the symmetric initial positions and eccentrically rotating in opposite directions. Here, it can be seen that horizontal movements in different widths occur in two massage tools, and there is no torsion.

FIG. 12 shows the operating radius structure of three adjacent massage tools in the first, second, third, and fourth massage tools 710, 720, 730 and 740 when eccentrically rotating in the same direction after aligning their initial positions with the center of the device. Here, it can be seen that vertical and horizontal movements over a large area and torsion occur in three massage tools.

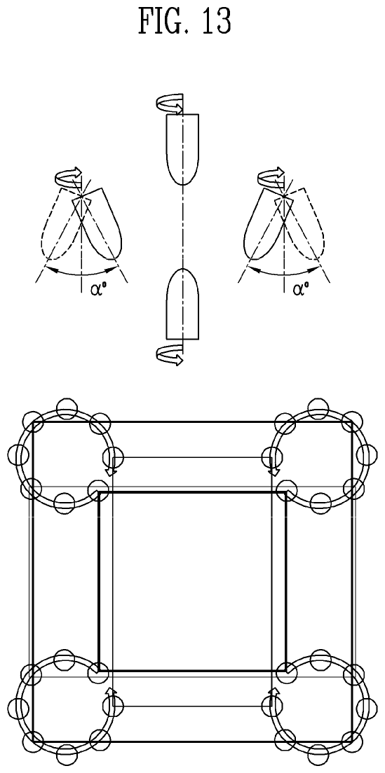

FIG. 13 shows the operating radius structure when the two facing massage tools eccentrically rotate in the forward direction, and the two other massage tools facing each other eccentrically rotate in the reverse direction, after all of the first, second, third and fourth massage tools 710, 720, 730 and 740 align their initial positions with the center of the device. Here, it can be seen that vertical and horizontal movements and torsion occur in a larger area than when there are three massage tools.

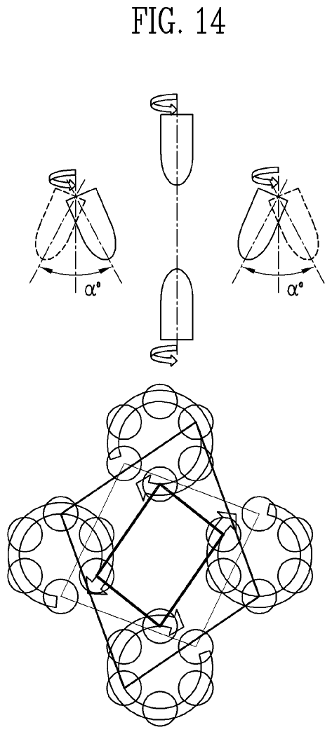

FIG. 14 shows the operating radius structure when all of the first, second, third, and fourth massage tools 710, 720, 730 and 740 eccentrically rotate in the same direction after aligning their initial positions with the center of the device. Here, it can be seen that vertical and horizontal movements and torsion occur in a larger area than when there are three massage tools. As such, when rotating after aligning the initial positions of the four massage tools 710, 720, 730 and 740 inclined by a predetermined angle with the center of the device, the massage effect and area change are diversified, and the vertical and horizontal movement and torsion effect are increased. In addition, it was confirmed that the massage feeling was particularly good when the initial position was aligned with the center of the device and then rotated eccentrically in the same direction. However, at the point where the eccentric massage tools 710, 720, 730 and 740 meet each other, a problem in which the body, hair, etc., are tightened and being pinched may occur, and such a problem can be solved by the operation of the abnormality detector 600 and the reverse unit 300 proposed in the present disclosure. This will be described in detail later along with the description of FIG. 7.

In the FIGS. 8 to 14, the eccentric angle of each of the first, second, third, and fourth massage tools 710, 720, 730 and 740 was set to α°, the eccentric rotation speed was the same, and the initial position, the eccentric rotation direction, and the number were varied to examine the operating radius, and as a result obtained from this, the massage function of kneading or rubbing can be said to be basic, and it can be seen that the cases in which the twisting massage function can be implemented together are the cases of FIGS. 10, 12, 13 and 14. As shown in FIGS. 13 and 14, the massage area and pattern vary according to the eccentric rotation direction of each of the plurality of massage tools. The user may select and change the eccentric rotation direction of the massage tool according to the massage mode.

As described above, according to the present disclosure, the massage area, pattern, and operating radius may vary depending on the initial position of the massage tool. Depending on embodiments, it may be configured to adjust the initial position according to the massage mode selected by the user. Various massage effects can be achieved by the user manually adjusting the massage tools before starting, using a button to make the initial positions of the massage tools the same, partially or completely symmetrically, or aligning the massage tools with the center of the device.

In addition, as shown in FIGS. 13 and 14, it can be seen that the greater the number of massage tools, the greater the operating radius.

In addition, the operating radius can be further increased even when the eccentric angle α° is increased or the length of the massage tool is increased.

This embodiment may be configured to implement more various massage area patterns in addition to the various massage area patterns having the operating radius of FIGS. 8 to 14, which will be described in detail below.

The first, second, third, and fourth massage tools 710, 720, 730 and 740 of this embodiment may be configured such that the eccentric rotation speed, the eccentric rotation direction, and the presence or absence of eccentric rotation are individually made. Here, the eccentric rotation speed is variable in three steps, for example, a first RPM, a second RPM greater than the first RPM, and a third RPM greater than the second RPM to perform the massage, but is not limited thereto.

Each of the first, second, third, and fourth massage tools 710, 720, 730 and 740 configures the gear built in the gearbox 530, for example, the transmission gear to correspond to each of the first, second, and third massage tools 710, 720, 730 and 740, and by controlling each of these transmission gears, the eccentric rotation speed and/or the eccentric rotation direction can be individually adjusted. In this embodiment, although the transmission gear is taken as an example, the eccentric rotation speed and/or the eccentric rotation direction of the first, second, third, and fourth massage tools 710, 720, 730 and 740 may be individually controlled by a combination of other gears.

In addition, each of the first, second, third, and fourth massage tools 710, 720, 730 and 740 may adjust the eccentric rotation speed individually by forming the first, second, third, and fourth shaft gears 551, 552, 553 and 554 of the shaft gear unit 550 in different gear ratios, or by allowing different stages to be selected by the first, second, third, and fourth clutches 541, 542, 543 and 544 by forming in at least two or more stages with gears having different gear ratios. If the RPM is different by changing the gear ratio of each eccentric rotation shaft, the operating area may be aperiodically deformed.

In addition, each of the first, second, third, and fourth massage tools 710, 720, 730 and 740 may individually regulate by controlling first, second, third, and fourth clutches 541, 542, 543 and 544 that individually regulate the power transmitted from the gearbox 530 to the first, second, third, and fourth drive shafts 561, 562, 563 and 564. In this way, the cosmetic device 1 of this embodiment may massage with 3 or 2 of the first, second, and third massage tools 710, 720, 730 and 740 instead of 4, thereby enabling the user to select various massages, by individually controlling the first, second, third, and fourth drive shafts 561, 562, 563 and 564 by the first, second, third, and fourth clutches 541, 542, 543 and 544.

As described above, the first, second, third and fourth massage tools 710, 720, 730 and 740 of this embodiment are configured such that the eccentric rotation speed, the eccentric rotation direction, and the presence or absence of eccentric rotation are individually made, it is possible to implement various aperiodic massage area patterns in addition to the various massage area patterns having the operating radius of FIGS. 8 to 14 described above. Accordingly, the cosmetic device 1 of the present embodiment may compositely implement a kneading massage function, a rubbing massage function, and a twisting massage function by the first, second, third, and fourth massage tools 710, 720, 730 and 740 in a one-time massage process.

In the first, second, third, and fourth massage tools 710, 720, 730 and 740 configured as described above, the massage area pattern is basically widened and narrowed, and when it is widened, if it is narrowed in a state where the finger is inserted, the finger is pressed and cannot be easily removed.

In this embodiment, in order to solve this problem, the first, second, third, and fourth massage tools 710, 720, 730 and 740 can be configured to have an auto-reverse function that rotates eccentrically at a certain angle in the opposite direction to release the restraint when the restraint by an external force occurs.

The auto-reverse function by the first, second, third, and fourth massage tools 710, 720, 730 and 740 may be implemented by the abnormality detector 600 and the reverse unit 300 as described above, it will be omitted herein in order to avoid duplicate description.

In addition, the first, second, third, and fourth massage tools 710, 720, 730 and 740 may be configured to implement an electric massage function together with a physical massage function.

The electric massage function by the first, second, third, and fourth massage tools 710, 720, 730 and 740 may be implemented by the pulse width modulator 400. When the electric massage function is selected in the input unit 20, the pulse width modulator 400 allows a minute current to flow through the first, second, third, and fourth massage tools 710, 720, 730 and 740, such as electronic muscle stimulation (EMS).

Here, the first, second, third, and fourth massage tools 710, 720, 730 and 740 are formed by applying a conductive material or conductive coating to perform an electric massage function by the pulse width modulator 400.

As shown in FIG. 15, the electric massage function may be performed in three steps.

For example, in step 1, the first electronic muscle stimulation is performed for a first time while varying the frequency in a state in which the first voltage is applied, in step 2, a second electronic muscle stimulation is performed for a second time while varying the frequency in a state where a second voltage higher than the first voltage is applied, and in step 3, the third electronic muscle stimulation is performed for a third time while varying the frequency in a state in which a third voltage higher than the second voltage is applied.

In the above, each of the first, second, and third times may be divided into first, second, and third sections (1t, 2t, 3t). Electronic muscle stimulation in each of the first, second, and third sections (1t, 2t, 3t) may be performed while varying the first, second, and third frequencies (AHz, BHz, CHz) for 1 second to 3 seconds. Here, the first, second, and third frequencies (AHz, BHz, CHz) may be varied within the range of 1 Hz to 15 Hz.

Thereafter, after stopping the electronic muscle stimulation for a predetermined time, the first electronic muscle stimulation of step 1 is performed again. Here, the predetermined time may be 1 second to 3 seconds.

The cosmetic device 1 according to this embodiment configured as described above can implement various functions such as a physical massage function, a physical+electric massage function, and an auto reverse function, and the present embodiment will be further understood by explaining the operation of the cosmetic device 1 with reference to FIG. 7.

Referring to FIG. 7, the input unit 20 turns “On” the power (step S1).

The input unit 20 selects a massage mode (physical massage mode/physical+electrical massage mode) and massage intensity (step S2).

According to the selection of the input unit 20, the first, second, third, and fourth massage tools 710, 720, 730 and 740 operate in the selected mode and intensity (step S3).

When the input unit 20 selects the physical massage mode, the main control unit 100 controls the rotation adjusting unit 200 to adjust the intensity according to the massage part. Here, the rotation adjusting unit 200 adjusts the eccentric rotation speed or eccentric rotation direction of the first, second, third, and fourth massage tools 710, 720, 730 and 740 in various ways, and accordingly, the intensity according to the massage part can be adjusted.

When the input unit 20 selects the electric massage mode in addition to the physical massage mode, the main control unit 100 controls the pulse width modulator 400 to adjust the intensity according to the massage part. Here, the pulse width modulator 400 variously adjusts the flow of fine currents such as electronic muscle stimulation (EMS) to the first, second, third, and fourth massage tools 710, 720, 730 and 740, and the intensity according to the massage part can be adjusted accordingly.

During the process of additionally performing a physical massage function or an electric massage function while the first, second, third, and fourth massage tools 710, 720, 730 and 740 rotate normally, it is determined whether the abnormal signal is transmitted to the main control unit 100 by the abnormality detector 600 (step S4). Here, when restraints caused by external forces occur in the first, second, third, and fourth massage tools 710, 720, 730 and 740, the eccentric rotation is limited and the motor 510 is overloaded, and when the generated overcurrent exceeds a preset value, the abnormal signal by the abnormal detector 600 is transmitted to the main control unit 100.

When an abnormal signal is not generated, the first, second, third, and fourth massage tools 710, 720, 730 and 740 operate normally (step S5), and thereafter, it is determined whether the normal operation time is a preset time, for example, 15 minutes (step S6).

When the normal operating time of the first, second, third, and fourth massage tools 710, 720, 730 and 740 is within the preset time range, the operation of the first, second, third, and fourth massage tools 710, 720, 730 and 740 is performed normally by returning to step S3, and when it exceeds the preset time range, the power is “OFF” to end the use of the cosmetic device 1 (step S13).

When an abnormal signal is generated, the reverse unit 300 receives the abnormal signal and checks the restraint state of the first, second, third, and fourth massage tools 710, 720, 730 and 740 for a first time (for example, 2 seconds) (step S7), stops the eccentric rotation of the first, second, third, and fourth massage tools 710, 720, 730 and 740 for a second time (for example, 2 seconds) (step S8), allows the eccentric rotation of the first, second, third, and fourth massage tools 710, 720, 730 and 740 to be eccentrically rotated in the reverse direction for a third time (for example, 1 second) (step S9), stops the eccentric rotation of the first, second, third, and fourth massage tools 710, 720, 730 and 740 again for a fourth time (for example, 2 seconds) (step S10), and allows the eccentric rotation of the first, second, third, and fourth massage tools 710, 720, 730 and 740 to be re-operated in forward rotation after eccentrically rotating in the reverse direction for a fifth time (for example, 1 second) (step S11).

The main control unit 100 determines whether the number of detections of the abnormal signal is a preset number, for example, 10 times (step S12), and when the number of abnormal signals is less than the preset number, the operation returns to step S3 and the first, second, third, and fourth massage tools 710, 720, 730 and 740 are normally operated, and when the number of abnormal signals reaches the preset number of times, the power is “OFF” to end the use of the cosmetic device 1 (step S13).

As such, this embodiment is configured to be able to individually control the eccentric rotation speed, eccentric rotation direction, and the presence or absence of eccentric rotation of each of the plurality of massage tools 710, 720, 730 and 740, and when the plurality of massage tools 710, 720, 730 and 740 rotate eccentrically while massaging the skin, by adding the massage area pattern to the basic area pattern, various non-periodic massage area patterns can be implemented, thereby maximizing massage efficiency.

In addition, in this embodiment, by configuring the plurality of massage tools 710, 720, 730 and 740 to have an auto-reverse function that rotates eccentrically at a certain angle in the opposite direction to release the restraint when the restraint by an external force occurs, damage to the device due to excessive overload or safety in use can be secured.

Although the present disclosure have been described above focusing on the embodiments, this is merely an example and does not limit the present disclosure, and one of ordinary skill in the art may understand that various combinations or modifications and applications not illustrated in the embodiments are possible without departing from the essential technical contents of the present embodiment. Accordingly, descriptions related to variations and applications that can be easily derived from the embodiments of the present disclosure should be interpreted as being included in the present disclosure.

<Explanation of Symbols>

| | |
|---|---|
| 1: Cosmetic device | 10: Body |
| 11: Housing | 12: Cover |
| 13: Handle | 20: Input unit |
| 30: Output unit | 40: Power unit |
| 50: First grip unit | 60: Second grip unit |
| 100: Main control unit | 200: Rotation adjusting unit |
| 300: Reverse unit | 400: Pulse width modulator |
| 500: Driving unit | 510: Motor |
| 520: Driving gear | 530: Gearbox |
| 540: Clutch unit | 541: First clutch |
| 542: Second clutch | 543: Third clutch |
| 544: Fourth clutch | 550: Shaft gear unit |
| 551: First shaft gear | 552: Second shaft gear |
| 553: Third shaft gear | 554: Fourth shaft gear |
| 560: Drive shaft unit | 561: First drive shaft |
| 562: Second drive shaft | 563: Third drive shaft |
| 564: Fourth drive shaft | 600: Abnormality detector |
| 700: Massage unit | 710: First massage tool |
| 720: Second massage tool | 730: Third massage tool |
| 740: Fourth massage tool | |

1. A cosmetic device which massages skin while a plurality of massage tools rotate eccentrically by operation of a motor, characterized by comprising:
an abnormality detector configured to generate an abnormal signal when an abnormality occurs in the motor or each of the plurality of massage tools; and
a reverse unit configured to operate according to the abnormal signal,
wherein the reverse unit is configured to perform an auto-reverse function of eccentrically rotating the eccentric rotation of the plurality of massage tools at a predetermined angle in an opposite direction.

2. The cosmetic device of claim 1, characterized in that the reverse unit is configured to:
receive the abnormal signal to check a restraint state of the plurality of massage tools for a first time;
stop the eccentric rotation of the plurality of massage tools for a second time;
cause the eccentric rotation of the plurality of massage tools to be eccentrically rotated in a reverse direction for a third time;
stop the eccentric rotation of the plurality of massage tools again for a fourth time; and
cause the eccentric rotation of the plurality of massage tools to be eccentrically rotated in a reverse direction for a fifth time and then the plurality of massage tools to be re-operated in forward rotation.

3. The cosmetic device of claim 1, characterized in that the plurality of massage tools are respectively connected to a transmission gear which is built in a gearbox connected to the motor and individually controlled, and are individually operated according to control of an eccentric rotation speed and an eccentric rotation direction of the transmission gear.

4. The cosmetic device of claim 3, characterized in that, when massaging skin while rotating eccentrically, the plurality of massage tools compositely implements a massage function of kneading, a massage function of rubbing, and a massage function of twisting in various non-periodic massage area patterns.

5. The cosmetic device of claim 3, characterized by further comprising:
a plurality of shaft gears to which the plurality of massage tools are coupled, respectively; and
a plurality of clutches provided separately between the gearbox and the plurality of shaft gears.

6. The cosmetic device of claim 5, characterized in that the plurality of massage tools are individually regulated by the plurality of clutches, and the regulated corresponding massage tools do not rotate eccentrically.

7. The cosmetic device of claim 5, characterized in that the plurality of shaft gears are formed in a same gear ratio, are formed in different gear ratios, or are formed in at least two or more stages with gears having different gear ratios.

8. The cosmetic device of claim 7, characterized in that each of the plurality of shaft gears is connected to each of the plurality of clutches, respectively,
when formed in the same gear ratio, the plurality of massage tools have the same eccentric rotation speed, and
when formed in the different gear ratios or formed in the at least two or more stages with gears having different gear ratios, the plurality of massage tools have different eccentric rotation speeds.

9. The cosmetic device of claim 1, characterized by further comprising:
a pulse width modulator configured to apply an electronic muscle stimulation (EMS) to the plurality of massage tools,
wherein the pulse width modulator is configured to control EMS intensity of the plurality of massage tools while varying a voltage and a frequency.

10. The cosmetic device of claim 9, characterized in that the pulse width modulator is configured to:
perform a first EMS for a first time while varying a frequency in a state where a first voltage is applied;
perform a second EMS for a second time while varying the frequency in a state where a second voltage higher than the first voltage is applied;
perform a third EMS for a third time while varying the frequency in a state where a third voltage higher than the second voltage is applied; and
perform the first EMS again after stopping the EMS for a predetermined period of time.

11. The cosmetic device of claim 1, characterized in that the plurality of massage tools are provided inclined by a predetermined angle, and
rotate eccentrically after their initial positions are aligned with a center of the device.
